(19) United States
(12) Patent Application Publication
(10) Pub. No.: US 2018/0079814 A1
(43) Pub. Date: Mar. 22, 2018

(54) COMBINED ANTI-PLD-1 AND ANTI-CTLA-4 ANTIBODIES FOR TREATING NON-SMALL LUNG CANCER

(71) Applicant: MedImmune Limited, Cambridge (GB)

(72) Inventors: Brandon W. Higgs, Gaithersburg, MD (US); Philip Z. Brohawn, Gaithersburg, MD (US); Wei Zhu, Gaithersburg, MD (US); Zheng Liu, Gaithersburg, MD (US); Jiaqi Huang, Gaithersburg, MD (US); Katie Streicher, Gaithersburg, MD (US); Yihong Yao, Gaithersburg, MD (US)

(21) Appl. No.: 15/562,247

(22) PCT Filed: Mar. 31, 2016
(86) PCT No.: PCT/EP2016/057075

§ 371 Date: Sep. 27, 2017

Related U.S. Application Data
(60) Provisional application No. 62/141,617, filed on Apr. 1, 2015.

(51) Int. Cl. C07K 16/28; C12Q 1/6886; A61P 35/00

(52) U.S. Cl. CPC C07K 16/2827; C12Q 1/6886; A61K 2039/545; A61P 35/00; C07K 2317/76; C07K 16/2818

(57) ABSTRACT

A method for treating lung cancer (e.g., non-small cell lung cancer) with an anti-PD-L1 antibody (MEDI4736), alone or in combination with an anti-CTLA4 antibody (Tremelimumab), in a patient identified using a polynucleotide marker of IFNgamma.

### BACKGROUND OF THE INVENTION

Lung cancer is among the most common forms of cancer and is the leading cause of cancer deaths among men and women. More people die of lung cancer annually than of colon, breast, and prostate cancers combined. Non-small cell lung cancer is the most common form of lung cancer. While the risk of acquiring lung cancer is higher among patients with a history of smoking, lung cancer also affects non-smokers. Improving survival of lung cancer patients remains difficult despite improved medical therapies. Most lung cancer is detected only in advanced stages when therapy options are limited. There is a growing recognition that lung cancer and other malignancies arise from a variety of pathogenic mechanisms. Methods of characterizing these malignancies at a molecular level is useful for stratifying patients, thereby quickly directing them to effective therapies. Improved methods for predicting the responsiveness of subjects having lung cancer, including NSCLC, are urgently required.

### SUMMARY OF THE INVENTION

The present invention provides methods for treating lung cancer (e.g., non-squamous cell or squamous cell non-small cell lung cancer) with an anti-PD-L1 antibody or a combination of an anti-PD-L1 antibody and an anti-CTLA4 antibody in a patient identified by detecting IFNgamma polynucleotide expression in a tumor or blood sample of the patient.

In one aspect, the invention features a method of treatment involving administering an anti-PD-L1 antibody, or an antigen binding fragment thereof, to a patient identified as having a squamous cell carcinoma or non-squamous cell carcinoma non-small cell lung cancer tumor that expresses an IFNgamma gene.

In another aspect, the invention features a method of treatment involving administering MEDI4736 or an antigen binding fragment thereof to a patient identified as having a squamous cell carcinoma or non-squamous cell carcinoma non-small cell lung cancer tumor that expresses an IFNgamma mRNA.

In another aspect, the invention features a method of identifying a subject having a squamous cell carcinoma or non-squamous cell carcinoma non-small cell lung cancer responsive to anti-PD-L1 therapy, the method involving detecting an increase in the level of IFNgamma gene expression in a non-small cell lung cancer tumor of the subject, relative to a reference, thereby identifying the non-small cell lung cancer as responsive to anti-PD-L1 therapy. In one embodiment, the IFNgamma gene expression is detected by real-time PCR.

In another aspect, the invention features a method of treatment involving administering a combination of an anti-PD-L1 antibody and an anti-CTLA4 antibody, or antigen binding fragments thereof, to a patient identified as having a squamous cell carcinoma or non-squamous cell carcinoma non-small cell lung cancer tumor that expresses IFNgamma. In one embodiment, the anti-PD-L1 antibody is MEDI4736 and the anti-CTLA4 antibody is Tremelimumab.

In another aspect, the invention features a method of treatment involving administering a combination of MEDI4736 and Tremelimumab, or antigen binding fragments thereof, to a patient identified as having a squamous cell carcinoma or non-squamous cell carcinoma non-small cell lung cancer tumor that expresses IFNgamma.

In another aspect, the invention features a method of identifying a subject having a squamous cell carcinoma or non-squamous cell carcinoma non-small cell lung cancer responsive to a combination of an anti-PD-L1 therapy and an anti-CTLA4 therapy, the method involving detecting an increase in the level of IFNgamma gene expression in a non-small cell lung cancer tumor of the subject, relative to a reference, thereby identifying the non-small cell lung cancer as responsive to an anti-PD-L1 therapy and an anti-CTLA4 therapy.

In various embodiments of the above aspects or any aspect of the invention described herein, the anti-PD-L1 antibody is MEDI4736. In other embodiments of the above aspects or any aspect of the invention described herein, the tumor expresses IFNgamma mRNA. In other embodiments of the above aspects or any aspect of the invention described herein, the patient is identified as responsive to MEDI4736. In other embodiments of the above aspects or any aspect of the invention described herein, Interferon gamma gene expression is detected in a Real-Time PCR assay. In other embodiments of the above aspects or any aspect of the invention described herein, the IFNgamma mRNA is detected in a blood sample. In other embodiments of the above aspects or any aspect of the invention described herein, at least about 0.1, about 0.3, about 1, about 3, about 10, about 15 mg/kg, or about 20 mg/kg MEDI4736, or an antigen-binding fragment thereof, is administered. In other embodiments of the above aspects or any aspect of the invention described herein, about 1 mg/kg MEDI4736, or an antigen-binding fragment thereof, is administered; about 3 mg/kg MEDI4736, or an antigen-binding fragment thereof, is administered; about 10 mg/kg MEDI4736 or an antigen-binding fragment thereof is administered; about 15 mg/kg MEDI4736, or an antigen-binding fragment, thereof is administered; or about 20 mg/kg MEDI4736, or an antigen-binding fragment, thereof is administered. In other embodiments of the above aspects or any aspect of the invention described herein, the administration is repeated about every 14, 21, or 28 days. In other embodiments of the above aspects or any aspect of the invention described herein, at least two doses is administered; at least three doses is administered; or at least five doses is administered.

In other embodiments of the above aspects or any aspect of the invention described herein, the patient is identified as responsive to MEDI4736 and Tremelimumab. In other embodiments of the above aspects or any aspect of the invention described herein, the marker is present in a tumor or blood sample. In other embodiments of the above aspects or any aspect of the invention described herein, at least 1 mg/kg, at least about 3 mg/kg, or about 10 mg/kg Tremelimumab, or an antigen-binding fragment thereof, is administered. In other embodiments of the above aspects or any aspect of the invention described herein, about 1 mg/kg Tremelimumab, or an antigen-binding fragment, thereof is administered; about 3 mg/kg Tremelimumab, or an antigen-binding fragment, thereof is administered; or about 10 mg/kg Tremelimumab, or an antigen-binding fragment, thereof is administered. In other embodiments of the above aspects or any aspect of the invention described herein, about 1 mg/kg Tremelimumab and about 20 mg/kg MEDI4736 or an antigen-binding fragment thereof is administered. In other embodiments of the above aspects or any aspect of the invention described herein, about 3 mg/kg Tremelimumab and about 15 mg/kg MEDI4736 or an antigen-binding fragment thereof is administered. In other embodiments of the above aspects or any aspect of the invention described herein, about 10 mg/kg Tremelimumab and about 15 mg/kg MEDI4736 or an antigen-binding fragment thereof is administered. In other embodiments of the above aspects or any aspect of the invention described herein, about 3 mg/kg Tremelimumab and about 10 mg/kg MEDI4736 or an antigen-binding fragment thereof is administered. In other embodiments of the above aspects or any aspect of the invention described herein, the administration is repeated about every 14, 21, or 28 days. In other embodiments of the above aspects or any aspect of the invention described herein, at least two, three, or five doses is administered.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Interferon gamma (IFNgamma) protein” is meant a polypeptide or fragment thereof having immunomodulatory activity. An exemplary IFNgamma amino acid sequence (Uniprot Accession No. P01579) is provided below:

>sp|P01579|IFNG_HUMAN Interferon gamma OS = Homo
sapiens GN = IFNG PE = 1 SV = 1
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ

By “IFNgamma polynucleotide” is meant a nucleic acid molecule encoding IFNgamma. The sequence of an exemplary IFNgamma polynucleotide is provided at NCBI Accession No. NM_000619, which is reproduced below:

| | |
|---|---|
| 1 | cacattgttc tgatcatctg aagatcagct attagaagag aaagatcagt taagtccttt |
| 61 | ggacctgatc agcttgatac aagaactact gatttcaact tctttggctt aattctctcg |
| 121 | gaaacgatga aatatacaag ttatatcttg gcttttcagc tctgcatcgt tttgggttct |
| 181 | cttggctgtt actgccagga cccatatgta aaagaagcag aaaaccttaa gaaatatttt |
| 241 | aatgcaggtc attcagatgt agcggataat ggaactcttt tcttaggcat tttgaagaat |
| 301 | tggaaagagg agagtgacag aaaaataatg cagagccaaa ttgtctcctt ttacttcaaa |
| 361 | ctttttaaaa actttaaaga tgaccagagc atccaaaaga gtgtggagac catcaaggaa |
| 421 | gacatgaatg tcaagttttt caatagcaac aaaaagaaac gagatgactt cgaaaagctg |
| 481 | actaattatt cggtaactga cttgaatgtc caacgcaaag caatacatga actcatccaa |
| 541 | gtgatggctg aactgtcgcc agcagctaaa acagggaagc gaaaaaggag tcagatgctg |
| 601 | tttcgaggtc gaagagcatc ccagtaatgg ttgtcctgcc tgcaatattt gaattttaaa |
| 661 | tctaaatcta tttattaata tttaacatta tttatatggg gaatatattt ttagactcat |
| 721 | caatcaaata agtatttata atagcaactt ttgtgtaatg aaaatgaata tctattaata |
| 781 | tatgtattat ttataattcc tatatcctgt gactgtctca cttaatcctt tgttttctga |
| 841 | ctaattaggc aaggctatgt gattacaagg ctttatctca ggggccaact aggcagccaa |
| 901 | cctaagcaag atcccatggg ttgtgtgttt atttcacttg atgatacaat gaacacttat |
| 961 | aagtgaagtg atactatcca gttactgccg gtttgaaaat atgcctgcaa tctgagccag |
| 1021 | tgctttaatg gcatgtcaga cagaacttga atgtgtcagg tgaccctgat gaaaacatag |
| 1081 | catctcagga gatttcatgc ctggtgcttc caaatattgt tgacaactgt gactgtaccc |
| 1141 | aaatggaaag taactcattt gttaaaatta tcaatatcta atatatatga ataaagtgta |
| 1201 | agttcacaac aaaaaaaaaa aaaaaaaaaa aaaaaaaaaa |

By “anti-PD-L1 antibody” is meant an antibody or antigen binding fragment thereof that selectively binds a PD-L1 polypeptide. Exemplary anti-PD-L1 antibodies are described for example at WO 2011/066389, which is herein incorporated by reference. MEDI4736 is an exemplary PD-L1 antibody. Following treatment with MEDI4736, a patient achieves disease control (DC). Disease control can be a complete response (CR), partial response (PR), or stable disease (SD). Sequences of MEDI4736 are provided in a sequence listing herein below.

By “CTLA4 nucleic acid molecule” is meant a polynucleotide encoding a CTLA4 polypeptide. An exemplary CTLA4 nucleic acid molecule sequence is provided at GenBank Accession No. AAL07473.

By “CTLA4 polypeptide” is meant a polypeptide having at least 85% amino acid sequence identity to GenBank Accession No. AAL07473.1 or a fragment thereof having T cell inhibitory activity. The sequence of AAL07473.1 is provided below:

gi|15778586|gb|AAL07473.1|AF414120_1 CTLA4
[Homo sapiens]
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

By “anti-CTLA4 antibody” is meant an antibody that selectively binds a CTLA4 polypeptide. Exemplary anti-CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences are provided below.

Tremelimumab U.S. Pat. No. 6,682,736

PSSLSASVGDRVTITCRASQSINSYLDWYQQKPGKAPKLLIYAASSLQS
GVPSRFSGSGSGTDFTLTISSLQPEDFATYYCQQYYSTPFTFGPGTKVE
IKRTVAAPSVFIFPPSDEQLKSGTASVVCLLNNFYPREAKV

### Tremelimumab VH

GVVQPGRSLRLSCAASGFTFSSYGMHWVRQAPGKGLEWVAVIWYDGSNKYY
ADSVKGRFTISRDNSKNTLYLQMNSLRAEDTAVYYCARDPRGATLYYYYYG
MDVWGQGTTVTVSSASTKGPSVFPLAPCSRSTSESTAALGCLVKDYFPEPV
TVSWNSGALTSGVH

### Tremelimumab VH CDR1

GFTFSSYGMH

### Tremelimumab VH CDR2

VIWYDGSNKYYADSV

### Tremelimumab VH CDR3

DPRGATLYYYYYGMDV

### Tremelimumab VL CDR1

RASQSINSYLD

### Tremelimumab VL CDR2

AASSLQS

### Tremelimumab VL CDR3

QQYYSTPFT

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≧50% relative to baseline. Confirmation can be obtained using a consecutive repeat assessment at least 4 weeks from the date of first documentation.

“Stable disease” (SD) indicates a decrease in tumor burden of 50% relative to baseline cannot be established and a 25% increase compared to nadir cannot be established.

By “PD-L1 polypeptide” is meant a polypeptide or fragment thereof having at least about 85%, 95% or 100% amino acid identity to NCBI Accession No. NP_001254635 and having PD-1 and CD80 binding activity.

| PD-L1 polypeptide sequence | |
|---|---|
| NCBI ACCESSION NO. NP_001254635 | |
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

By “PD-L1 nucleic acid molecule” is meant a polynucleotide encoding a PD-L1 polypeptide. An exemplary PD-L1 nucleic acid molecule sequence is provided at NCBI Accession No. NM_001267706.

| PD-L1 nucleic acid sequence | |
|---|---|
| NCBI ACCESSION NO. NM_001267706 mRNA | |
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind PD-L1 or CTLA4 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}) however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “biologic sample” is meant any tissue, cell, fluid, or other material derived from an organism. In one embodiment, a biological sample is a tumor biopsy sample.

A “biomarker” or “marker” as used herein generally refers to a protein, nucleic acid molecule, clinical indicator, or other analyte that is associated with a disease. In one embodiment, a marker is differentially present in a biological sample obtained from a subject having a disease (e.g., lung cancer) relative to the level present in a control sample or reference.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Lung cancer includes small cell lung cancer (SCLC) and non-small cell lung cancer (NSCLC). There are three main subtypes of NSCLC: squamous cell carcinoma, adenocarcinoma, and large cell (undifferentiated) carcinoma. Other subtypes include adenosquamous carcinoma and sarcomatoid carcinoma.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison. In one embodiment, the level of interferon gamma present in a sample from a patient that is partially responsive to a therapy of the invention is compared to the level present in a corresponding sample obtained from a patient having progressive disease.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a bar graph that shows interferon gamma gene expression at baseline in tumor and blood samples obtained from patients with squamous cell carcinoma (SCC) non-small cell lung cancer (NSCLC). The patients were subsequently treated with an anti-PD-L1 antibody. Subjects were characterized as having SCC NSCLC that was confirmed or unconfirmed as having a partial response (PR) to anti-PD-L1 treatment, as having stable disease (SD), as having progressive disease (PD) or as not evaluable (NE).

FIG. 2 provides two scatter plots showing baseline tumor and blood interferon gamma (IFGN mRNA) expression in patients subsequently treated with an anti-PD-L1 antibody. dCT indicates the difference in interferon gamma expression prior to and following anti-PD-L1 antibody therapy. Sens=sensitivity; Spec=specificity; PPV=positive predictive value; NPV=negative predictive value; PR=partial responder; PD=progressive disease; NE=not evaluable.

FIG. 3 provides a scatter plot and two bar graphs showing a correlation between baseline tumor and blood interferon gamma mRNA expression in patients subsequently treated with an anti-PD-L1 antibody.

FIG. 4 provides two scatter plots showing tumor size change and dCT IFNgamma mRNA in partial responder (PR) patients having non-squamous cell carcinoma non-small cell lung cancer (SCC NSCLC) treated with a combination of anti-CTLA4 and anti-PD-L1 antibodies. Dosages for the MEDI4736 and Tremelimumab (“Treme”) are shown in the bottom panel: Treme 1 mg/kg, MEDI4736 20 mg/kg Q4 W, Treme 3 mg/kg, MEDI4736 15 mg/kg Q4w; Treme 10 mg/kg, MEDI4736 15 mg/kg Q4 W; Treme 3 mg/kg, MEDI4736 10 mg/kg Q2W.

FIG. 5 includes two pie charts showing clinical response status of interferon gamma mRNA positive and negative patients treated with a combination of anti-CTLA4 and anti-PD-L1 antibodies.

### LIST OF SEQUENCES

MEDI4736 light chain variable region amino acid sequence: SEQ ID NO:1

MEDI4736 heavy chain variable region amino acid sequence: SEQ ID NO:2.

MEDI4736 heavy chain variable region amino acid sequence of CDR1, CDR2, and CDR3: SEQ ID NOs:3-5.

MEDI4736 light chain variable region amino acid sequence of CDR1, CDR2, and CDR3: SEQ ID NOs:6-8.

### DETAILED DESCRIPTION OF THE INVENTION

The present invention provides methods for treating lung cancer (e.g., non-squamous cell or squamous cell non-small cell lung cancer) with an anti-PD-L1 antibody or a combination of an anti-PD-L1 antibody and an anti-CTLA4 antibody in a patient identified by detecting IFNgamma polynucleotide expression in a tumor or blood sample of the patient.

The invention is based, at least in part, on the discovery that patients having lung cancer (e.g., non-squamous cell or squamous cell non-small cell lung cancer) that is responsive to treatment with an anti-PD-L1 antibody or a combination of an anti-PD-L1 antibody and an anti-CTLA4 antibody may be identified by detecting increased levels of interferon gamma mRNA in a tumor or blood sample. Accordingly, the invention provides methods for identifying subjects that have lung cancer that is likely to respond to anti-PD-L1 antibody treatment, alone or in combination with an anti-CTLA4 antibody, based on the presence or level of IFNgamma mRNA in a subject tumor or blood sample.

### B7-H1/PD-L1

B7-H1, also known as PD-L1, is a type I transmembrane protein of approximately 53 kDa in size. In humans B7-H1 is expressed on a number of immune cell types including activated and anergic/exhausted T cells, on naïve and activated B cells, as well as on myeloid dendritic cells (DC), monocytes and mast cells. It is also expressed on non-immune cells including islets of the pancreas, Kupffer cells of the liver, vascular endothelium and selected epithelia, for example airway epithelia and renal tubule epithelia, where its expression is enhanced during inflammatory episodes. B7-H1 expression is also found at increased levels on a number of tumours including, but not limited to breast, colon, colorectal, lung, renal, including renal cell carcinoma, gastric, bladder, non-small cell lung cancer (NSCLC), hepatocellular cancer (HCC), and pancreatic cancer, as well as melanoma.

B7-H1 is known to bind two alternative ligands, the first of these, PD-1, is a 50-55 kDa type I transmembrane receptor that was originally identified in a T cell line undergoing activation-induced apoptosis. PD-1 is expressed on activated T cells, B cells, and monocytes, as well as other cells of the immune system and binds both B7-H1 (PD-L1) and the related B7-DC (PD-L2). The second is the B7 family member B7-1, which is expressed on activated T cells, B cells, monocytes and antigen presenting cells.

Signaling via the PD-1/B7-H1 axis is believed to serve important, non-redundant functions within the immune system, by negatively regulating T cell responses. B7-H1 expression on tumor cells is believed to aid tumors in evading detection and elimination by the immune system. B7-H1 functions in this respect via several alternative mechanisms including driving exhaustion and anergy of tumour infiltrating T lymphocytes, stimulating secretion of immune repressive cytokines into the tumour micro-environment, stimulating repressive regulatory T cell function and protecting B7-H1 expressing tumor cells from lysis by tumor cell specific cytotoxic T cells.

### Anti-PD-L1 Antibodies

Antibodies that specifically bind and inhibit PD-L1 activity (e.g., binding to PD-1 and/or CD80) are useful for the treatment of lung cancer (e.g., non-small cell lung cancer

MEDI4736 is an exemplary anti-PD-L1 antibody that is selective for B7-H1 and blocks the binding of B7-H1 to the PD-1 and CD80 receptors. MEDI4736 can relieve B7-H1-mediated suppression of human T-cell activation in vitro and inhibits tumor growth in a xenograft model via a T-cell dependent mechanism. Other agents that could be used include agents that inhibit PD-L1 and/or PD-1 (AB or other).

Information regarding MEDI4736 (or fragments thereof) for use in the methods provided herein can be found in International Application Publication No. WO 2011/066389 A1, the disclosure of which is incorporated herein by reference in its entirety. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a specific aspect, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in WO 2011/066389 A1, which is herein incorporated by reference in its entirety.

### CTLA4 Blocking Antibody: Tremelimumab

Accordingly, in one embodiment therapeutic combinations of the invention comprise a CTLA4 blocking antibody (e.g., Tremelimumab) and/or antibodies that reduce PD1/PD-L1 interactions. Two T cell modulatory pathways receiving significant attention to date signal through cytotoxic T lymphocyte antigen-4 (CTLA4, CD152) and programmed death ligand 1 (PD-L1, also known as B7H-1 or CD274).

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed primarily on T cells, and the expression of its ligands CD80 (B71) and CD86 (B7.2), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma but did not significantly increase the overall survival of patients compared to the standard of care (temozolomide or dacarbazine) at that time.

Information regarding tremelimumab (or antigen-binding fragments thereof) for use in the methods provided herein can be found in U.S. Pat. No. 6,682,736 (where it is referred to as 11.2.1), the disclosure of which is incorporated herein by reference in its entirety. Tremelimumab (also known as CP-675,206, CP-675, CP-675206, and ticilimumab) is a human IgG2 monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region and a heavy chain variable region. In a specific aspect, tremelimumab or an antigen-binding fragment thereof for use in the methods provided herein comprises a heavy chain variable region and a light chain variable region identified herein. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

Characterizing Responsiveness to Anti-PD-L1 Antibody and/or Anti-CTLA4 Therapy

In characterizing the responsiveness of lung cancer in a subject to an anti-PD-L1 antibody treatment alone or in combination with an anti-CTLA4 antibody treatment, the level of IFNgamma expression is measured in different types of biologic samples (e.g., tumor or blood samples).

IFNgamma polynucleotide expression is higher in a tumor or blood sample obtained from a subject that is responsive to anti-PD-L1 antibody treatment or treatment with a combination of the invention comprising an anti-PD-L1 antibody and an anti-CTLA4 antibody than the level of expression in a non-responsive subject (e.g., a subject with progressive disease). In one embodiment, an alteration in expression is calculated using −ΔCt, where Ct represents cycle threshold values. For example, the −ΔCtvalue of an IFNgamma gene is obtained and from that value the Ct value of a reference gene (e.g., B2M, ACTB, GAPDH) is subtracted from the mean Ct value for IFNgamma gene to obtain a Delta-Ct value. Then this value is multiplied by −1 to retain directionality. In other embodiments, expression of a marker of the invention is increased by at least about 2, 3, 4, 5 or 10-fold in a responsive patient relative to the level in a non-responsive subject (e.g., a subject with progressive disease). IFNgamma polynucleotide fold change values are determined using any method known in the art, including but not limited to quantitative PCR, RT-PCR, Northern blotting, in situ hybridization, fluorescence in situ hybridization (FISH) and/or RNA sequencing.

In particular embodiments, the responsiveness of lung cancer in a subject to anti-PD-L1 antibody treatment or treatment with a combination of the invention comprising an anti-PD-L1 antibody and an anti-CTLA4 antibody, is assayed by detecting IFNgamma expression (e.g., mRNA expression).

### Selection of a Treatment Method

Subjects suffering from lung cancer (e.g., squamous or non-squamous cell carcinoma non-small cell lung cancer) may be tested for IFNgamma polynucleotide expression in the course of selecting a treatment method. Patients characterized as having high expression (e.g., as defined by Ct score) or increased expression relative to a reference level are identified as responsive to anti-PD-L1 treatment or treatment with a combination of the invention comprising an anti-PD-L1 antibody and an anti-CTLA4 antibody.

Treatment with an Anti-PD-L1 Antibody

Patients identified as having tumors or blood samples that express IFNgamma, particularly at high levels, are likely to be responsive to anti-PD-L1 antibody therapy or treatment with a combination of the invention comprising an anti-PD-L1 antibody and an anti-CTLA4 antibody. Such patients are administered an anti-PD-L1 antibody, such as MEDI4736, or an antigen-binding fragment thereof, alone or in combination with Tremelimumab. MEDI4736, alone or in combination with Tremelimumab, or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

In some embodiments, at least two doses of MEDI4736, alone or in combination with Tremelimumab, or an antigen-binding fragment thereof, are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, MEDI4736, alone or in combination with Tremelimumab, or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, MEDI4736, alone or in combination with Tremelimumab, or an antigen-binding fragment thereof is administered over a three-week treatment period, a six-week treatment period, over a nine-week treatment period, over a twelve-week treatment period, over a twenty-four-week treatment period, or over a one-year or more treatment period. In some embodiments, MEDI4736, alone or in combination with Tremelimumab, or an antigen-binding fragment thereof is administered over a two-month treatment period, over a four-month treatment period, or over a six-month or more treatment period (e.g., during a maintenance phase).

The amount of MEDI4736, alone or in combination with Tremelimumab, or an antigen-binding fragment thereof to be administered to the patient will depend on various parameters, such as the patient's age, weight, clinical assessment, tumor burden and/or other factors, including the judgment of the attending physician.

In certain aspects the patient is administered one or more doses of MEDI4736 or an antigen-binding fragment thereof wherein the dose is about 0.1 mg/kg. In certain aspects the patient is administered one or more doses of MEDI4736 or an antigen-binding fragment thereof wherein the dose is about 0.3 mg/kg. In certain aspects the patient is administered one or more doses of MEDI4736 or an antigen-binding fragment thereof wherein the dose is about 1 mg/kg. In certain aspects the patient is administered one or more doses of MEDI4736 or an antigen-binding fragment thereof wherein the dose is about 3 mg/kg. In certain aspects the patient is administered one or more doses of MEDI4736 or an antigen-binding fragment thereof wherein the dose is about 10 mg/kg. In certain aspects the patient is administered one or more doses of MEDI4736 or an antigen-binding fragment thereof wherein the dose is about 15 mg/kg. In certain aspects the patient is administered one or more doses of MEDI4736 or an antigen-binding fragment thereof wherein the dose is about 20 mg/kg MEDI4736, or an antigen-binding fragment thereof.

In certain aspects the patient is administered at least two doses of MEDI4736 or an antigen-binding fragment thereof wherein the dose is about 0.1 mg/kg. In certain aspects the patient is administered at least two doses of MEDI4736 or an antigen-binding fragment thereof wherein the dose is about 0.3 mg/kg. In certain aspects the patient is administered at least two doses of MEDI4736 or an antigen-binding fragment thereof wherein the dose is about 1 mg/kg. In certain aspects the patient is administered at least two doses of MEDI4736 or an antigen-binding fragment thereof wherein the dose is about 3 mg/kg. In certain aspects the patient is administered at least two doses of MEDI4736 or an antigen-binding fragment thereof wherein the dose is about 10 mg/kg. In certain aspects the patient is administered at least two doses of MEDI4736 or an antigen-binding fragment thereof wherein the dose is about 15 mg/kg. In certain aspects the patient is administered at least two doses of MEDI4736 or an antigen-binding fragment thereof wherein the dose is about 20 mg/kg. In some embodiments, the at least two doses are administered about two weeks apart. In some embodiments, the at least two doses are administered about three weeks apart. In some embodiments, the at least two doses are administered about four weeks apart.

In certain aspects the patient is administered at least three doses of MEDI4736 or an antigen-binding fragment thereof wherein the dose is about 0.1 mg/kg. In certain aspects the patient is administered at least three doses of MEDI4736 or an antigen-binding fragment thereof wherein the dose is about 0.3 mg/kg. In certain aspects the patient is administered at least three doses of MEDI4736 or an antigen-binding fragment thereof wherein the dose is about 1 mg/kg. In certain aspects the patient is administered at least three doses of MEDI4736 or an antigen-binding fragment thereof wherein the dose is about 3 mg/kg. In certain aspects the patient is administered at least three doses of MEDI4736 or an antigen-binding fragment thereof wherein the dose is about 10 mg/kg. In certain aspects the patient is administered at least three doses of MEDI4736 or an antigen-binding fragment thereof wherein the dose is about 15 mg/kg. In certain aspects the patient is administered at least two doses of MEDI4736 or an antigen-binding fragment thereof wherein the dose is about 20 mg/kg. In some embodiments, the at least three doses are administered about two weeks apart. In some embodiments, the at least three doses are administered about three weeks apart. In some embodiments, the at least three doses are administered about four

In certain aspects, administration of MEDI4736, alone or in combination with Tremelimumab, or an antigen-binding fragment thereof according to the methods provided herein is through parenteral administration. For example, MEDI4736, alone or in combination with Tremelimumab, or an antigen-binding fragment thereof can be administered by intravenous infusion or by subcutaneous injection. In some embodiments, the administration is by intravenous infusion. In certain aspects the patient is administered one or more doses of Tremelimumab or an antigen-binding fragment thereof wherein the dose is about 1 mg/kg. In certain aspects the patient is administered one or more doses of Tremelimumab or an antigen-binding fragment thereof wherein the dose is about 3 mg/kg. In certain aspects the patient is administered one or more doses of Tremelimumab or an antigen-binding fragment thereof wherein the dose is about 10 mg/kg.

In certain aspects, MEDI4736, alone or in combination with Tremelimumab, or an antigen-binding fragment thereof is administered according to the methods provided herein in combination or in conjunction with additional cancer therapies. Such therapies include, without limitation, chemotherapeutic agents such as Vemurafenib, Erlotinib, Afatinib, Cetuximab, Carboplatin, Bevacizumab, Erlotinib, or Pemetrexed, or other chemotherapeutic agents, as well radiation or any other anti-cancer treatments.

The methods provided herein can decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects provided herein, the administration of MEDI4736, alone or in combination with Tremelimumab, can reduce a tumor size by at least 25%. In certain aspects provided herein, the administration of MEDI4736 alone or in combination with Tremelimumab, can reduce a tumor size by at least 25% within about 6 weeks of the first treatment. In certain aspects provided herein, the administration of MEDI4736, alone or in combination with Tremelimumab, can reduce a tumor size by at least 50%. In certain aspects provided herein, the administration of MEDI4736, alone or in combination with Tremelimumab, can reduce a tumor size by at least 50% within about 10 weeks of the first treatment. In certain aspects provided herein, the administration of MEDI4736, alone or in combination with Tremelimumab, can reduce a tumor size by at least 75%. In certain aspects provided herein, the administration of MEDI4736, alone or in combination with Tremelimumab, can reduce a tumor size by at least 75% within about 10 weeks of the first treatment.

In certain aspects, use of the methods provided herein, i.e., administration of MEDI4736, alone or in combination with Tremelimumab, or an antigen-binding fragment thereof can decrease tumor size within 6 weeks, within 7 weeks, within 8 weeks, within 9 weeks, within 10 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks of the first treatment.

In some embodiments, administration of 1 mg/kg of MEDI4736, alone or in combination with Tremelimumab, or an antigen-binding fragment thereof (e.g., at least one dose, at least two doses, at least three doses, at least four doses, at least five doses, at least six doses, at least seven doses, at least eight doses, at least nine doses, at least ten doses, or more every two weeks or every three weeks) can be sufficient to reduce tumor size. However, as provided herein, larger doses can also be administered, for example, to optimize efficacy, number of doses necessary, or certain pharmacokinetic parameters.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

In certain aspects, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≧50% relative to baseline. Confirmation can be obtained using a consecutive repeat assessment at least 4 weeks from the date of first documentation

“Progressive disease” (PD) refers to an increase in tumor burden ≧25% relative to the minimum recorded (nadir). Confirmation can be obtained by a consecutive repeat assessment at least 4 weeks from the date of first documentation. New, non-measurable lesions do not define PD.

“Stable disease” (SD) refers to not meeting the criteria for CR, PR, or PD.

In certain aspects, administration of MEDI4736, alone or in combination with Tremelimumab, or an antigen-binding fragment thereof can increase progression-free survival (PFS).

In certain aspects, administration of MEDI4736, alone or in combination with Tremelimumab, or an antigen-binding fragment thereof can increase overall survival (OS).

According to the methods provided herein, administration of MEDI4736, alone or in combination with Tremelimumab, or an antigen-binding fragment thereof can result in desirable pharmacokinetic parameters. Total drug exposure can be estimated using the “area under the curve” (AUC). “AUC (tau)” refers to AUC until the end of the dosing period, whereas “AUC (inf)” refers to the AUC until infinite time. The administration can produce AUC (tau) of about 100 to about 2,500 d·μg/mL. The administration can produce a maximum observed concentration (Cmax) of about 15 to about 350 μg/mL. The half-life of the MEDI4736 or the antigen-binding fragment thereof can be about 5 to about 25 days. In addition, the clearance of the MEDI4736 or the antigen-binding fragment thereof can be about 1-10 ml/day/kg.

As provided herein, MEDI4736 or an antigen-binding fragment thereof can also decrease free B7-H1 levels. Free B7-H1 refers to B7-H1 that is not bound (e.g., by MEDI4736). In some embodiments, B7-H1 levels are reduced by at least 80%. In some embodiments, B7-H1 levels are reduced by at least 90%. In some embodiments, B7-H1 levels are reduced by at least 95%. In some embodiments, B7-H1 levels are reduced by at least 99%. In some embodiments, B7-H1 levels are eliminated following administration of MEDI4736 or an antigen-binding fragment thereof. In some embodiments, administration of MEDI4736 or an antigen-binding fragment thereof reduces the rate of increase of B7-H1 levels as compared, e.g., to the rate of increase of B7-H1 levels prior to the administration of MEDI4736 or an antigen-binding fragment thereof.

### Kits

The invention provides kits for characterizing the responsiveness of a subject to anti-PD-L1 antibody treatment or treatment with a combination of the invention comprising an anti-PD-L1 antibody and an anti-CTLA4 antibody. In one embodiment, the kit includes a therapeutic composition containing an effective amount of an antibody that specifically binds a PD-L1 polypeptide in unit dosage form alone or in combination with an anti-CTLA4 antibody.

A diagnostic kit of the invention provides a reagent (e.g., TaqMan primers/probes for an IFNgamma polynucleotide and housekeeping reference genes) for measuring relative expression of an IFNgamma polynucleotide.

In some embodiments, the kit comprises a sterile container which contains a therapeutic and/or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In one embodiment, a kit of the invention comprises reagents for measuring an IFNgamma polynucleotide expression and a therapeutic anti-PD-L1 antibody or a combination of an anti-PD-L1 antibody and an anti-CTLA4 antibody. If desired, the kit further comprises instructions for measuring an IFNgamma polynucleotide expression and/or instructions for administering the anti-PD-L1 antibody or a combination of an anti-PD-L1 antibody and an anti-CTLA4 antibody to a subject having a lung cancer (e.g., squamous cell or non-squamous cell carcinoma non-small cell lung cancer) selected as responsive to anti-PD-L1 antibody treatment or treatment with a combination of the invention comprising an anti-PD-L1 antibody and an anti-CTLA4 antibody. In particular embodiments, the instructions include at least one of the following: description of the therapeutic agent; dosage schedule and administration for treatment or prevention of lung cancer (e.g., non-small cell lung cancer, small cell lung cancer) or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Interferon Gamma mRNA in Blood Predicts Clinical Response to Anti-PD-L1 Antibody Treatment in Squamous Cell Carcinomaa Non-Small Cell Lung Cancer (SCC NSCLC)

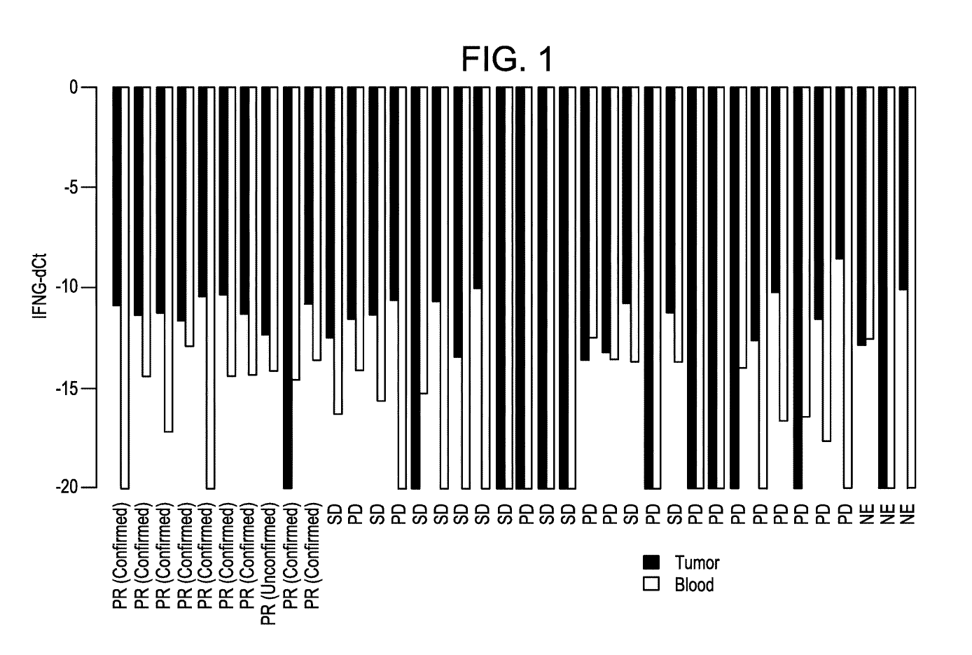

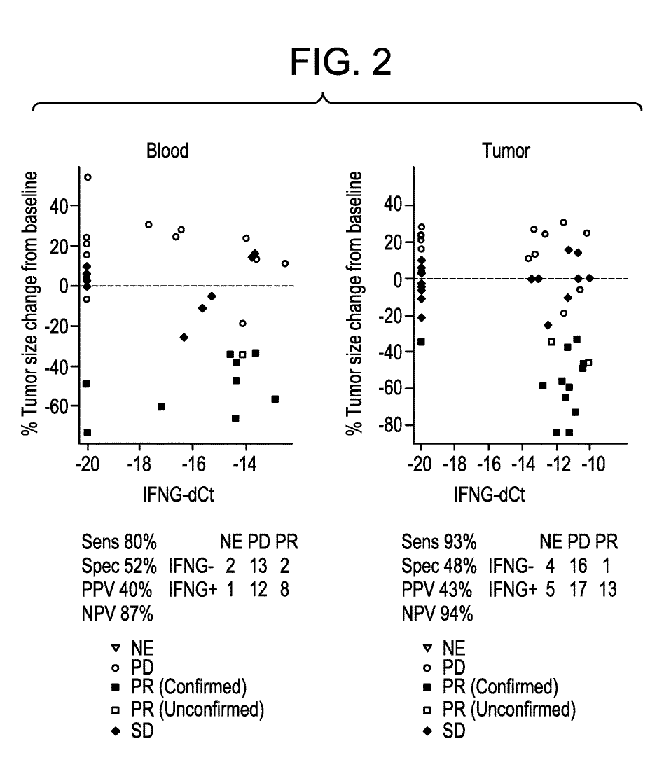

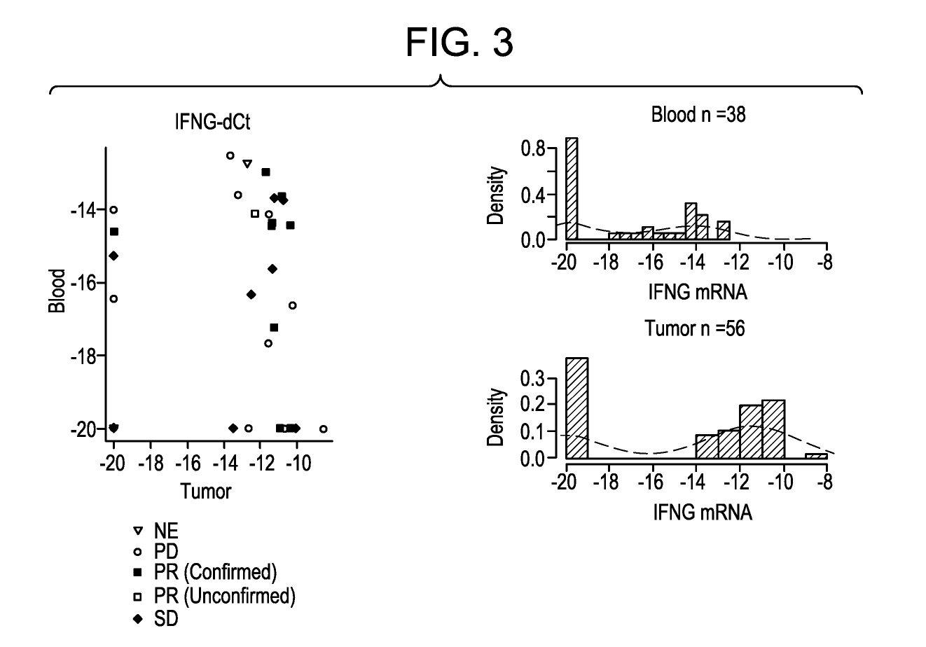

Interferon gamma (IFNγ) mRNA in blood showed a positive trend that indicated that it likely predicts clinical response to anti-PD-L1 antibody treatment in squamous cell carcinoma non-small cell lung cancer (SCC NSCLC). Baseline levels of interferon gamma expression in blood and tumor are shown in FIGS. 1 and 2. Levels of interferon gamma mRNA correlate in blood and tumor indicating that either can be used to evaluate clinical responsiveness to anti-PD-L1 antibody treatment. Patients that respond to anti-PD-L1 therapy have higher interferon gamma expression in blood and tumor than SCC NSCLC patients who do not respond to anti-PD-L1 therapy. The trend with tumor shrinkage is also prevalent among those patients with higher interferon gamma expression in both blood and tumor. FIG. 3 shows that levels of interferon gamma mRNA in tumor and in blood had a modest correlation. In sum, IFNγ mRNA in blood shows positive predictive value for anti-PD-L1 antibody (MEDI4736) clinical response in SCC NSCLC.

### Example 2: IFNγ mRNA is Predictive of Clinical Response for Anti-PD-L1 and Anti-CTLA4 Combination Therapy in Non-SCC NSCLC

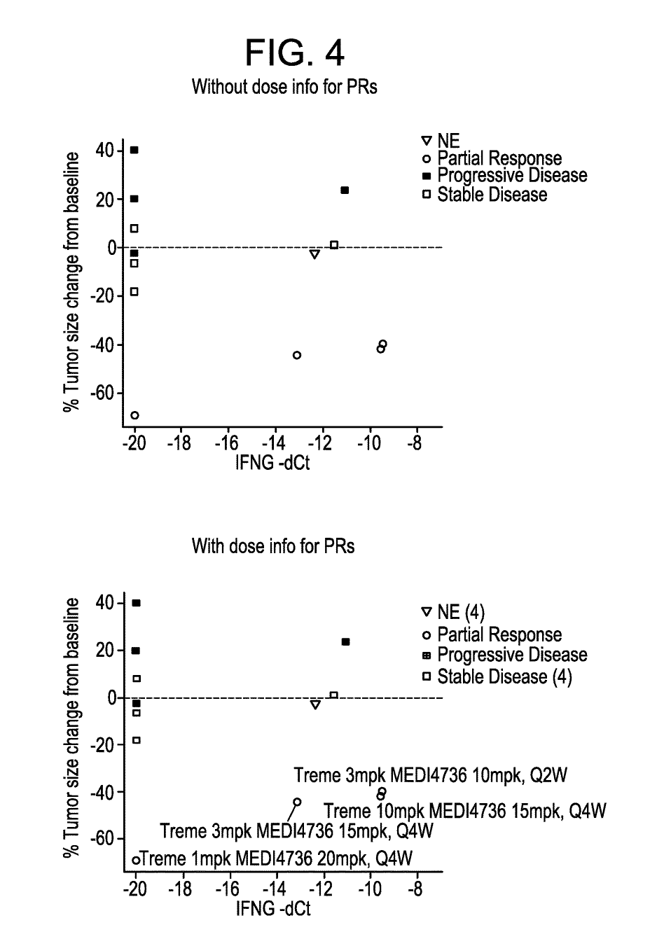

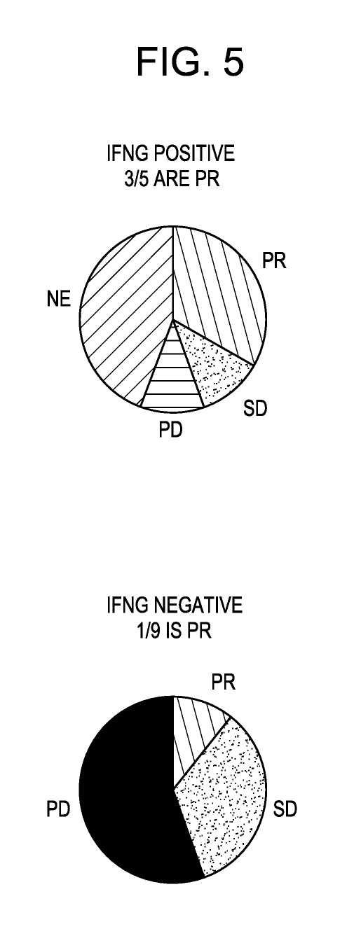

Nineteen patients have baseline high quality IFNγ mRNA to evaluate for predictive value. Eighteen patients have non squamous cell carcinoma non-small cell lung cancer (NSCC NSCLC, FIG. 4). The remaining patient does not have histology information available. Similar bimodal distribution of interferon gamma mRNA is observed in non-SCC NSCLC, similar to that observed in patients treated with anti-PD-L1 for NSCLC of the same histology. Of the nine non-squamous cell non-small cell lung cancer patients that are interferon gamma mRNA high, five have evaluable clinical outcome and three are partially responsive (PRs). One, three or ten mg/kg (mpk) of an anti-CTLA4 antibody (tremelimumab “treme”) was administered in combination with ten, fifteen, or twenty mg/kg of an anti-PD-L1 antibody (MEDI4736) (bottom panel). Doses were administered at two or four week intervals. Interestingly, three out of five partially responsive patients were interferon gamma positive at baseline while only one in nine partially responsive patients was interferon gamma negative (FIG. 5). In sum, IFNγ mRNA is predictive of clinical response for anti-PD-L1 and anti-CTLA4 combination therapy in non-SCC NSCLC.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Sequences for an illustrative example an anti-
PDL1 antibody, MEDI4736.

>PCT/US2010/058007_77 Sequence 77 from PCT/US2010/
058007 Organism: Homo sapiens
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
>PCT/US2010/058007_72 Sequence 72 from PCT/US2010/
058007 Organism: Homo sapiens
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
MEDI4736 VH CDR1
GFTFSRYWMS
MEDI4736 VH CDR2
NIKQDGSEKYYVDSVKG
MEDI4736 VH CDR3
EGGWFGELAFDY
MEDI4736 VL CDR1
RASQRVSSSYLA
MEDI4736 VL CDR2
DASSRAT
MEDI4736 VL CDR3
QQYGSLPWT

What is claimed is:

1. A method of treatment comprising administering an anti-PD-L1 antibody, or an antigen binding fragment thereof, to a patient identified as having a squamous cell carcinoma or non-squamous cell carcinoma non-small cell lung cancer tumor that expresses an IFNgamma gene.

2. The method of claim 1, wherein the anti-PD-L1 antibody is MEDI4736.

3. The method of any of claims 1-2, wherein the tumor expresses IFNgamma mRNA.

4. A method of treatment comprising administering MEDI4736 or an antigen binding fragment thereof to a patient identified as having a squamous cell carcinoma or non-squamous cell carcinoma non-small cell lung cancer tumor that expresses an IFNgamma mRNA.

5. The method of any of claims 1-4, wherein the patient is identified as responsive to MEDI4736.

6. The method of any of claims 1-5, wherein interferon gamma gene expression is detected in a Real-Time PCR assay.

7. The method of any of claims 1-6, wherein the IFNgamma mRNA is detected in a blood sample.

8. The method of any of claims 1-7, wherein at least about 0.1, about 0.3, about 1, about 3, about 10, about 15 mg/kg, or about 20 mg/kg MEDI4736, or an antigen-binding fragment thereof, is administered.

9. The method of claim 8, wherein about 1 mg/kg MEDI4736, or an antigen-binding fragment thereof, is administered.

10. The method of claim 8, wherein about 3 mg/kg MEDI4736, or an antigen-binding fragment thereof, is administered.

11. The method of claim 8, wherein about 10 mg/kg MEDI4736 or an antigen-binding fragment thereof is administered.

12. The method of claim 8, wherein about 15 mg/kg MEDI4736, or an antigen-binding fragment, thereof is administered.

13. The method of claim 8, wherein about 20 mg/kg MEDI4736, or an antigen-binding fragment, thereof is administered.

14. The method of any one of claims 1-8, wherein the administration is repeated about every 14, 21, or 28 days.

15. The method of any one of claims 1-8, wherein at least two doses is administered.

16. The method of any one of claims 1-8, wherein at least three doses is administered.

17. The method of any one of claims 1-8, wherein at least five doses is administered.

18. A method of identifying a subject having a squamous cell carcinoma or non-squamous cell carcinoma non-small cell lung cancer responsive to anti-PD-L1 therapy, the method comprising detecting an increase in the level of IFNgamma gene expression in a non-small cell lung cancer tumor of the subject, relative to a reference, thereby identifying said non-small cell lung cancer as responsive to anti-PD-L1 therapy.

19. The method of claim 18, wherein the IFNgamma gene expression is detected by real-time PCR.

20. A method of treatment comprising administering a combination of an anti-PD-L1 antibody and an anti-CTLA4 antibody, or antigen binding fragments thereof, to a patient identified as having a squamous cell carcinoma or non-squamous cell carcinoma non-small cell lung cancer tumor that expresses IFNgamma.

21. The method of claim 20, wherein the anti-PD-L1 antibody is MEDI4736 and the anti-CTLA4 antibody is Tremelimumab.

22. A method of treatment comprising administering a combination of MEDI4736 and Tremelimumab, or antigen binding fragments thereof, to a patient identified as having a squamous cell carcinoma or non-squamous cell carcinoma non-small cell lung cancer tumor that expresses IFNgamma.

23. The method of any one of claims 20-22, wherein the patient is identified as responsive to MEDI4736 and Tremelimumab.

24. The method of any one of claims 20-22, wherein interferon gamma gene expression is detected in a Real-Time PCR assay.

25. The method of any one of claims 20-22, wherein interferon gamma mRNA is present in a tumor or blood sample.

26. The method of any one of claims 20-22, wherein at least about 0.1, about 0.3, about 1, about 3, about 10, about 15 mg/kg, or about 20 mg/kg MEDI4736, or an antigen-binding fragment thereof, is administered.

27. The method of claim 26, wherein about 1 mg/kg MEDI4736, or an antigen-binding fragment thereof, is administered.

28. The method of claim 26, wherein about 3 mg/kg MEDI4736, or an antigen-binding fragment thereof, is administered.

29. The method of claim 26, wherein about 10 mg/kg MEDI4736 or an antigen-binding fragment thereof is administered.

30. The method of claim 26, wherein about 15 mg/kg MEDI4736, or an antigen-binding fragment, thereof is administered.

31. The method of claim 26, wherein about 20 mg/kg MEDI4736, or an antigen-binding fragment, thereof is administered.

32. The method of any of claims 20-31, wherein at least 1 mg/kg, at least about 3 mg/kg, or about 10 mg/kg Tremelimumab, or an antigen-binding fragment thereof, is administered.

33. The method of claim 32, wherein about 1 mg/kg Tremelimumab, or an antigen-binding fragment, thereof is administered.

34. The method of claim 32, wherein about 3 mg/kg Tremelimumab, or an antigen-binding fragment, thereof is administered.

35. The method of claim 32, wherein about 10 mg/kg Tremelimumab, or an antigen-binding fragment, thereof is administered.

36. The method of claim 32, wherein about 1 mg/kg Tremelimumab and about 20 mg/kg MEDI4736 or an antigen-binding fragment thereof is administered.

37. The method of claim 32, wherein about 3 mg/kg Tremelimumab and about 15 mg/kg MEDI4736 or an antigen-binding fragment thereof is administered.

38. The method of claim 32, wherein about 10 mg/kg Tremelimumab and about 15 mg/kg MEDI4736 or an antigen-binding fragment thereof is administered.

39. The method of claim 32, wherein about 3 mg/kg Tremelimumab and about 10 mg/kg MEDI4736 or an antigen-binding fragment thereof is administered.

40. The method of any one of claims 20-39, wherein the administration is repeated about every 14, 21, or 28 days.

41. The method of any of claims 32-40, wherein at least two doses is administered.

42. The method of any of claims 32-40, wherein at least three doses is administered.

43. The method of any of claims 32-40, wherein at least five doses is administered.

44. A method of identifying a subject having a squamous cell carcinoma or non-squamous cell carcinoma non-small cell lung cancer responsive to a combination of an anti-PD-L1 therapy and an anti-CTLA4 therapy, the method comprising detecting an increase in the level of IFNgamma gene expression in a non-small cell lung cancer tumor of the subject, relative to a reference, thereby identifying said non-small cell lung cancer as responsive to an anti-PD-L1 therapy and an anti-CTLA4 therapy.

45. The method of any one of claims 32-44, wherein the markers are detected by real-time PCR.


SEQUENCE LISTING (21 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Homo sapiens
GFTFSRYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

SEQ ID NO: 9 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 10 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 11 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 12 - protein, 139 aa, Artificial Sequence
  source: /note=“Description of Artificial Sequence Synthetic polypeptide”
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 13 - protein, 167 aa, Artificial Sequence
  source: /note=“Description of Artificial Sequence Synthetic polypeptide”
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 14 - protein, 10 aa, Artificial Sequence
  source: /note=“Description of Artificial Sequence Synthetic peptide”
GFTFSSYGMH

SEQ ID NO: 15 - protein, 15 aa, Artificial Sequence
  source: /note=“Description of Artificial Sequence Synthetic peptide”
VIWYDGSNKYYADSV

SEQ ID NO: 16 - protein, 16 aa, Artificial Sequence
  source: /note=“Description of Artificial Sequence Synthetic peptide”
DPRGATLYYYYYGMDV

SEQ ID NO: 17 - protein, 11 aa, Artificial Sequence
  source: /note=“Description of Artificial Sequence Synthetic peptide”
RASQSINSYLD

SEQ ID NO: 18 - protein, 7 aa, Artificial Sequence
  source: /note=“Description of Artificial Sequence Synthetic peptide”
AASSLQS

SEQ ID NO: 19 - protein, 9 aa, Artificial Sequence
  source: /note=“Description of Artificial Sequence Synthetic peptide”
QQYYSTPFT

SEQ ID NO: 20 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 21 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa